(19) United States
(12) Patent Application Publication
(10) Pub. No.: US 2008/0208055 A1
(43) Pub. Date: Aug. 28, 2008

(54) Method and Device for the Sonographic Navigated Repositioning of Bone Fragments

(76) Inventors: Michael Bertram, Markt Schwaben (DE); Georg Maier, Munchen (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/026,900

(22) Filed: Feb. 6, 2008

Related U.S. Application Data
(60) Provisional application No. 60/895,501, filed on Mar. 19, 2007.

(30) Foreign Application Priority Data
Feb. 7, 2007 (EP) 07101887.3

(51) Int. Cl. A61B 8/00; A61B 17/68

(52) U.S. Cl. 600/443; 606/300

(57) ABSTRACT

A method and device for aligning the first and second fragments of a fractured bone relative to each other, wherein the bone fragments may be aligned using images from an ultrasound device that is tracked by a navigation system. The positions and/or orientations of landmarks on each of the bone fragments may be used to set the bone alignment for more precise healing.

### RELATED APPLICATION DATA

This application claims priority of U.S. Provisional Application No. 60/895,501 filed on Mar. 19, 2007, and EP 07 101 887.3 filed on Feb. 7, 2007, which are incorporated herein by reference.

### FIELD OF THE INVENTION

The present invention relates to a method and system for using sonographic images for navigated alignment of dislocated fractures.

### BACKGROUND OF THE INVENTION

When operatively treating a fracture, it is advantageous to precisely align and position the fragments created by the fracture relative to each other. Precise alignment of the fragments relative to each other enables the fragments to heal and fuse in accordance with the natural anatomical conditions. If the fragments of a fractured femur are not correctly positioned relative to each other and are fixed or set while twisted relative to each other, this can lead to complications. The complications can include a false posture or restricted movement and/or excessive strain on the patient's joint structures due to a significant change in the natural anatomical structure and biomechanics. An anatomically twisted position of the proximal and distal structures forming the joint can be described in the case of the femur by a so-called antetorsion angle (the angle relative to each other along the shaft axis). This angle should be restored in the event of fractures to the femur. Other bones also can have a rotational deviation from their anatomical longitudinal axis after fractures, and these deviations should be corrected when taking care of the fracture.

In clinically known methods, landmarks are recorded in the articular region far from the fracture, using various methods—in most cases, imaging methods—and the landmarks are aligned with respect to each other. In a known method, images of the femoral head (the greater trochanter and the femoral condyles) are used for determining the femoral antetorsion angle. To align the fragments, the known methods either use the anatomical mean values, or the anatomical conditions on the healthy side are examined and transferred onto the fractured side.

Problems can occur when both sides are affected by fractures and there is no healthy side available as a guide. On the other hand, problems can occur because the anatomical conditions on one side cannot be directly transferred onto the other side. Since most people do not have an exactly symmetrical bone structure, the values for femoral antetorsion vary significantly within a group. Consequently, inaccuracies and misplacements can occur using known methods, even when a healthy side is available as a guide, since only rarely are the anatomical conditions of the two sides exactly identical.

WO 2005/115246 A1 relates to a method and device for non-invasively determining prominent structures in the human or animal body. The reference discloses a method wherein the body may be irradiated with ultrasound radiation in the area of the prominent structure using an ultrasound—emitting and receiving—head that receives or detects ultrasound radiation reflected at or by the prominent structure. The system then represents on a display an image that corresponds to the transit time of the reflected radiation. In order to determine the position of the prominent structure in the body, the method discloses determining the position and orientation of the ultrasound head and of the body in the area of the prominent structure using a navigation system. The navigation system uses marking elements fixed to the ultrasound head and the body and discloses the following steps:
selecting a prominent structure on the display,
determining the position and orientation of the prominent structure relative to the ultrasound head using the radiating direction of the ultrasound head and the measured transit time of the ultrasound radiation, and
determining the position and orientation of the prominent structure relative to the body from the position and orientation of the ultrasound head relative to the body.

U.S. Pat. No. 6,379,302 relates to superimposing navigation information onto ultrasound images, wherein a surgical navigation system includes an ultrasound machine and a computer connected to the ultrasound machine. The software may be loaded into a memory of the computer and contains instructions that, when they are carried out by the computer, cause the computer to generate an icon that represents a surgical instrument having a tip and a trajectory. The computer can superimpose the icon on a real-time ultrasound image that has an image plane such that when the surgical instrument intersects the image plane, the format of the trajectory of the surgical instrument may be changed to represent the surgical instrument intersecting the ultrasound image plane. The system also includes a localizer connected to the ultrasound machine, and a display connected to the computer for displaying the generated icon superimposed on the real-time image.

### SUMMARY OF THE INVENTION

In a method in accordance with the invention, a user may ascertain alignment information for the treatment of fractured bones. The bones that may be treated include a femur, tibia, humerus, scapula, clavicle, calcaneum, metatarsal bone, metacarpal bone, navicular bone, or another similar bone. In order to restore the anatomy and correct biomechanics of the bone, the bone fragments can be aligned or positioned relative to each other such that they can heal in a correct position relative to each other.

In general, the method may include the following. An ultrasound device can be positioned with respect to the part of the body on which the fracture is situated, such that areas in a region around the fracture can be detected using the ultrasound device. The ultrasound device can detect characteristic landmarks of the bone, situated on the circumference or outer side of the bone. Before the landmarks are detected, the operator can determine which landmarks on the bone are to be detected by the ultrasound device. The landmarks may be situated in a region around the fracture of the bone. The landmarks can be situated a few millimeters to a few centimeters away from the fracture. It may be preferable to avoid landmarks on the ends of the respective bone facing away from the fracture. In other words, local landmarks in relation to the fracture are detected and sonographically represented, and their alignment may be used in the method.

In order to record the landmarks, the ultrasound device may be positioned with respect to the bone such that the ultrasound device detects at least one characteristic landmark on a first fragment and the corresponding characteristic landmark on a second fragment. The characteristic landmarks on the first and second fragments can be identified in the ultrasonic recordings of the bone.

A tracking reference may be arranged on the ultrasound device. The tracking reference may be a reference star, which may emit or reflect infrared radiation, or another suitable device. Using the tracking reference, a navigation system can track the ultrasound device, such that the spatial position and/or orientation of the ultrasound device can be ascertained. The navigation system may include or be connected to a computational unit. The navigation system and/or computational unit can determine or ascertain the positions and/or orientations of the landmarks with respect to the ultrasound device. A tracking reference (such as a reference star) can be arranged on each of the first fragment of the bone and the second fragment of the bone. Using the tracking reference, the spatial position and/or orientation of the first and second fragments of the bone can be ascertained by the navigation system. Thus, the position and/or orientation of the detected characteristic landmarks can be ascertained with respect to the reference stars arranged on the bone fragments and, relative to the bone. The spatial positions and/or orientations of the first and second bone fragments, the ultrasound device, and the landmarks can be ascertained or calculated.

Based on the ascertained position and/or orientation of the landmarks on the first and second fragments, alignment information can be ascertained and outputted or communicated that provides guidance for aligning the first and second fragments relative to each other. Absolute positional information can be ascertained using coordinates or absolute positional values with respect to a defined spatial coordinate system. This positional information can provide guidance for the correct alignment and positioning for the first and second bone fragments. This information also may indicate relative placement parameters such as differential values in multiple degrees of freedom between the fragments, which may provide guidance for positioning one of the fragments in order to correctly align the fragment relative to the other fragment. This information also may be used to ascertain and provide information on changes in alignment. For example, the information may be used to provide guidance for changing the relative positioning the first and second fragments in order to correctly align the fragments relative to each other.

The first and second fragments of a fractured bone may be aligned relative to each other by rotating the first and second fragments around a longitudinal axis of the bone. An intramedullary pin also can be introduced or inserted into the medullary channel, around which the fragments can be rotated about the longitudinal axis of the bone. Additionally, translational movements of the fragments and other rotational movements around other axes also can be performed in order to position the fragments relative to each other.

The first and second fragments of the bone can be aligned relative to each other such that the characteristic landmark on the first bone fragment and the characteristic landmark on the second bone fragment lie in a predetermined or ascertainable positional relationship relative to each other. A user can provide or enter the positional relationship in which the characteristic landmarks are to lie. For example, a user can provide that the landmarks are to be positioned opposing each other, or a user can provide the direction and modulus that describe the distance or positional relationship of the landmarks relative to each other. Based on the provided or predetermined positional relationship of the landmarks relative to each other, the first and second fragments of the bone can be aligned relative to each other. The computational unit also can ascertain or propose useful positional relationships of the landmarks relative to each other, from which a user can select the desired positional relationship. The bone fragments can be positioned relative to each other based on a selected positional relationship or multiple proposed positional relationships.

The linea aspera of the femur may be used as a characteristic landmark on the first and second fragment of a fractured bone. The linea aspera is a longitudinally running crest on the dorsal upper leg bone, and consists of a lateral lip and a medial lip (labium). The linea aspera may be used as a landmark when the fracture lies within an area in which it may be particularly easy to recognize (particularly, in the middle third of the femur). The linea aspera also can be automatically proposed as a possible landmark by the computational unit if it is “known” or in the memory of the computational unit. Moreover, it may be provided or entered into the computational unit that the fracture lies in the middle area, in particular in the middle third, of the femur. When the fracture lies in the middle area of the femur, the ultrasound device can be moved manually or automatically near the fracture, to record as landmarks the linea aspera on the first and second fragments of the bone. The linea aspera can be identified or recognized in the ultrasound recordings manually by a user, semi-automatically, or automatically using image recognition algorithms. By taking into account the ascertained positions and/or orientations of the ultrasound device and the fragments, the position and/or orientation of the linea aspera can be ascertained. By taking into account the position and/or orientation of the linea aspera, the computational unit can ascertain and provide guidance for moving and aligning the linea aspera on the first fragment and the linea aspera on the second fragment relative to each other to ensure that the fragments are correctly aligned. Alignment information can be provided by the computational unit to provide guidance for moving or rotating the fragments relative to each other, so that the linea aspera on the first fragment transitions directly into the linea aspera on the second fragment. In other words, the fragments should be aligned such that the linea aspera on the bone formed from the first and second fragments forms a continuous relatively straight line with no kinks or discontinuities. Aligning the fragments in this way can ensure that the torsion angle between fragments is small or at least minimized, so that the bone can heal in its original anatomical position.

Other characteristic bone structures, such as lines, curves, depressions or edges, also can be used as characteristic landmarks instead of the linea aspera.

On the femur, the transverse elliptical cross-sectional shape of the distal metaphysis or the eccentric cross-section of the ridge of the trochanter can be used. Other landmarks that can be used include front edge of the tibia, the spiral groove, the distal epicondyles of the humerus or, depending on the structure of a fracture, even the line of fracture in the bone itself.

A user may provide or select ultrasound device placement information that describes the position at which and/or the orientation with which the ultrasound device may be navigated or moved using the navigation system. The ultrasound device may detect the landmark on the first fragment from a first position, and the landmark on the second fragment from a second position. It is possible to provide a position that lies in the longitudinal direction of the bone or along the longitudinal axis of the bone, in a particular region around the fracture. The particular region could be an area having a diameter of equal to or less than one-half centimeter or up to five or more centimeters. It is possible to provide an orientation such that the ultrasound device approaches a perpendicular relation to the bone or the part of the body containing the bone. The position and shape of the fracture determines whether landmarks in the area of the fracture can be used. If the fracture is situated in an area where the landmarks may be automatically defined, the ultrasound device may be positioned accordingly. The surgeon can intra-operatively define prominent bone structures as local landmarks. The nearer the landmark is to the fracture, the lower the resultant distortion. If the fracture does not lie on or near characteristic landmarks, the position of the ultrasound device may be approximated. Based on the ultrasound device placement information and using the navigation system, the ultrasound device can be moved manually or automatically to the provided position and orientation.

It is also possible to align more than two fragments of a bone relative to each other using a method in accordance with the invention. By aligning and positioning three or more fragments, using the same methodology described above, a bone with more than one fracture may be precisely set for proper healing.

Once the fragments have been aligned relative to each other, they can be connected to each other at their connecting point by an intramedullary pin inserted into or located at the medullary channel. The intramedullary pin can be inserted into the femur, from the hip end into an upper part of the femur or from the knee end into a lower part of the femur. The intramedullary pin can be positioned such that the intramedullary pin lies in the medullary channel of the first and second fragments of the bone in such a way that the fragments can be connected to each other via the intramedullary pin. The position of the intramedullary pin can be ascertained using x-ray recordings, for example using a C-arm. Based on the x-ray recordings, the computational unit can ascertain the intramedullary pin's positioning in the medullary channel and provide guidance for changing its position. The computational unit may provide guidance for navigating the intramedullary pin into place in the medullary channel. The intramedullary pin may include fixing elements, such as holes or bores, which run transverse or perpendicular to the longitudinal axis or to the medullary channel of the bone and via which the intramedullary pin can be fixed. Insertion elements such as rods or screws may be used in connection with the fixing elements, such that the intramedullary pin can be fixed relative to the bone fragments. Fixing the fragments relative to each other using fixing elements may enable, simplify, and/or expedite the healing process.

The method described herein may be at least partially implemented via a computer that executes a computer program. The computer program can be provided on a computer readable medium such that when executed by the computer, carries out the method in accordance with the invention.

A device in accordance with the invention includes an ultrasound device and a navigation system including a computational unit such as a computer or processor. The computational unit can be integrated into the navigation system or can be connected to the navigation system wirelessly or via a wire connection. A tracking reference, such as a reference star which reflects or emits infrared radiation, can be arranged on the ultrasound device and can be detected by the navigation system, such that the navigation system can ascertain a spatial position and/or orientation of the tracking reference and therefore also of the ultrasound device. Using ultrasound radiation, the ultrasound device can record ultrasound images of a part of the body and of a bone lying in the part of the body. A tracking reference, such as a reference star, which can be detected by the navigation system, can be arranged on each of the fragments created by the fracture to the bone, such that the navigation system can ascertain the spatial position and/or orientation of the reference stars and therefore of the fragments.

The ultrasound device can be moved, manually, semi-automatically or automatically, to a position from which it can record an area of the first fragment. Within this area, a characteristic landmark may be situated on the circumference or outer side of the first fragment. This landmark can be the linea aspera of the femur or another characteristic landmark. On the femur, this landmark can be the distal metaphysis or the cross-section of the ridge of the trochanter. Other landmarks that can be used include the front edge of the tibia, the spiral groove, the distal epicondyles of the humerus or, depending on the structure of a fracture, even the line of fracture in the bone itself. The area recorded by the ultrasound device may be situated near the fracture, such as a few millimeters or centimeters away from the fracture. Once the area of the first fracture has been recorded, the ultrasound device can be moved manually, semi-automatically or automatically to another position from which it can record an area of the second fragment of the bone. This area should include at least one homogenous or heterogeneous characteristic landmark situated on the circumference or outer side of the second fragment. The second landmark can be homogenous with the first landmark, e.g., the linea aspera can be recorded on both fragments. The ultrasound device, however, also can record heterogeneous characteristic landmarks. Based on the ascertained position and/or orientation of the ultrasound device and the ascertained position and/or orientation of the fragments of the bone, the computational unit can ascertain the spatial position and/or orientation of the landmarks. Moreover, the computational unit can ascertain position and orientation of the landmarks with respect to the bone. The computational unit can “know,” or have in memory, the position and/or orientation of the landmark on the circumference of the first fragment relative to the first fragment, and the position and/or orientation of the landmark on the circumference of the second fragment relative to the second fragment. Based on the ascertained position and/or orientation of the landmark on the first fragment, and the ascertained position and/or orientation of the landmark on the second fragment, the computational unit can ascertain alignment information that provides guidance for aligning the fragments. Such guidance may be used to precisely align the fragments relative to each other, or at least align the fragments to obtain as small a rotational error or axial deviation as possible.

The device in accordance with the invention can also include a display device connected to the ultrasound device. The ultrasound images recorded can be represented on the display device. The ascertained alignment information also can be displayed on the display device. The alignment information can be superimposed on the ultrasound images and can provide guidance for changing the relative positioning of fragments. The alignment information can be displayed on the display device as numerical values or as coordinates. The alignment information also can be represented graphically, for example as arrows, which provide visual guidance for moving or changing the relative positioning of the fragments.

The navigation system may be designed such that the ultrasound device can be navigated to particular positions to record the landmarks of the first and second bone fragments. A user can manually provide or select the ultrasound device positions by providing coordinates or positional values. The computational unit also can provide ultrasound device placement information such as recommended positions and orientations for recording the landmarks.

### BRIEF DESCRIPTION OF THE DRAWINGS

The forgoing and other features of the invention are hereinafter discussed with reference to the drawing.

FIG. 1 illustrates an exemplary system in accordance with the present invention comprising an ultrasound probe positioned at two positions for recording the linea aspera of the femur as a landmark.

### DETAILED DESCRIPTION

In the following detailed description, a hip joint is described as an example of an anatomical joint, wherein the range of motion of the hip joint, more specifically the given range of motion of the (anatomical) hip joint of a patient, may be used to determine a suitable orientation of the artificial joint.

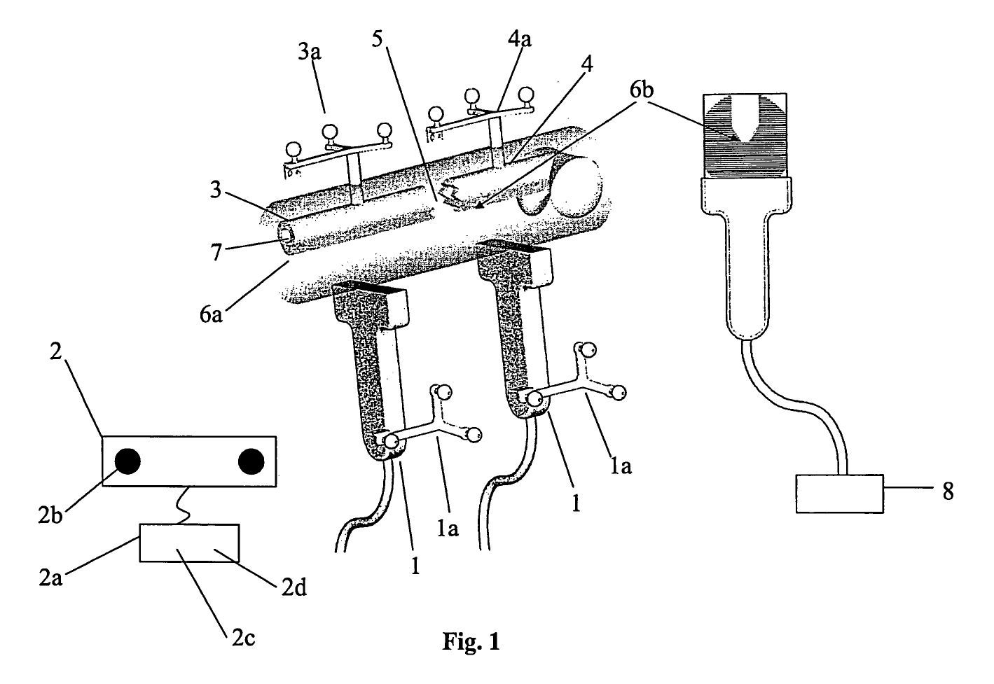

FIG. 1 shows an exemplary system in accordance with the present invention including: an ultrasound probe; a navigation system 2 that may include infrared cameras 2b; and a computer 2a (which includes a processor 2c and memory 2d) connected to the navigation system 2. A reference star 1a can be arranged on the ultrasound probe 1 and can be detected by the navigation system 2, such that the computer 2a can ascertain the position and orientation of the reference star 1a connected to the ultrasound probe 1 and therefore also the position and orientation of the ultrasound probe 1. FIG. 1 also shows a femur 5 that has been broken apart into two fragments 3, 4 by a fracture. Reference stars 3a, 4a can be respectively arranged on each of the two fragments 3, 4. The navigation system 2 can detect the reference stars 3a, 4a arranged on the fragments 3, 4, such that the computer 2a can ascertain the position and orientation of the reference star 3a connected to the first fragment 3 of the bone, and the position and orientation of the reference star 4a connected to the second fragment 4 of the bone, and therefore also the position and orientation of the first and second fragments 3, 4 of the bone.

The ultrasound probe 1 can be initially positioned almost perpendicular to the femur in a first position in which the ultrasound probe 1 can record the first fragment of the bone 3 and, as a landmark, the linea aspera 6a arranged on the circumference or outer side of the first fragment of the bone 3. Once the probe 1 has recorded these features, the ultrasound probe 1 can be positioned almost perpendicular to the femur in a second position in which the ultrasound probe 1 can record the second fragment of the bone 4 and, as a landmark, the linea aspera 6b arranged on the circumference or outer side of the second fragment of the bone 4.

The linea aspera 6a, 6b of the first and second fragments 3, 4 of the bone can be identified in the ultrasound recordings, such that by taking into account the ascertained positions and orientations of the fragments 3, 4, and of the ultrasound device 1, the computational unit 2a can ascertain the position and orientation of the linea aspera 6a of the first fragment of the bone 3 and the linea aspera 6b of the second fragment of the bone 4. If the computational unit 2a knows, or has in its memory from a manual input, a predetermined criteria (i.e. dimensions, angles, tolerances, ranges, guidelines, or other user selected parameters) for positioning the linea aspera 6a, 6b of the two fragments 3, 4 relative to each other, the computational unit 2a can use:
the position and orientation of the ultrasound probe 1;
the positions and orientations of the fragments 3, 4; and
the criteria used for the desired positions of the linea aspera 6a, 6b relative to each other,
   to determine alignment information for guiding changes to the relative positions and orientations of the fragments 3, 4. A user can provide criteria requiring that the linea aspera 6a of the first fragment of the bone 3 be transitioned directly into the linea aspera 6b of the second fragment of the bone 4. By taking into account this criteria, and by taking into account the positions and orientations of the fragments 3, 4, and of the ultrasound device 1, the computational unit 2a can ascertain angular values that can be displayed on a screen 8, such that the fragments 3, 4 may be rotated by the displayed angular values to change the positions of the fragments 3, 4 relative to each other.

Once the two fragments 3, 4 have been aligned relative to each other, an intramedullary pin (not shown) may be inserted into the medullary channel 7 of the femur. The intramedullary pin may penetrate both fragments though the medullary channel 7 of the first and second fragments 3, 4. The intramedullary pin may include transverse bores or transverse holes that receive fastening elements. The fastening elements cooperate with the transverse holes and the femur to fixes the fragments 3, 4 relative to the intramedullary pin and therefore, also relative to each other. With the bone fragments correctly aligned relative to each other, they may heal properly.

The method corrects or fixes the antetorsion angle such that the fragments 3, 4 heal and fuse together to form a femur which has a greater correspondence to the original, natural anatomical shape of the femur, as compared to a femur restored using conventional methods.

Although the invention has been shown and described with respect to a certain preferred embodiment or embodiments, it is obvious that equivalent alterations and modifications will occur to others skilled in the art upon the reading and understanding of this specification and the annexed Figures. In particular regard to the various functions performed by the above described elements (components, assemblies, devices, software, computer programs, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for aligning a first fragment and a second fragment of a fractured bone relative to each other, comprising:
using an ultrasound device to identify at least one landmark on each of the first fragment of the bone and the second fragment of the bone;
determining a spatial position and/or orientation of each of the identified landmarks; and
using the determined spatial position and/or orientation of each of the identified landmarks to determine an alignment of the first and second fragments of the bone that satisfies a predetermined criteria.

2. The method according to claim 1, wherein determining the spatial positions and/or orientations of the identified landmarks includes:
determining a spatial position and/or orientation of the ultrasound device; and of the first and second fragments; and
using the determined spatial positions and/or orientations of the ultrasound device and the first and second bone fragments and data obtained from the ultrasound device during a scan of the first and second bone fragments to determine the spatial positions and/or orientations of the landmarks.

3. The method according to claim 2, wherein determining a spatial position and/or orientation of the ultrasound device; and of the first and second fragments determining a spatial position and/or orientation of the ultrasound device; and of the first and second fragments includes:
arranging a tracking reference on the ultrasound device;
arranging respective tracking devices on each of the first and second fragments; and
using a navigation system to track and ascertain the positions and/or orientations of the ultrasound device and of the first and second fragments using the tracking references arranged on each of the ultrasound device and first and second fragments.

4. The method according to claim 1, wherein the fractured bone is a femur, tibia, humerus or fibula.

5. The method according to claim 1, further comprising:
aligning the first and second fragments of the bone relative to each other such that the at least one landmark on each of the first fragment of the bone and the second fragment of the bone lie in a predetermined positional relationship relative to each other.

6. The method according to claim 1, further comprising:
aligning the first and second fragments of the bone relative to each other by rotating them around a longitudinal axis of the bone or around a medullary channel of the bone.

7. The method according to claim 1, wherein the identified landmarks are located on the circumferences of the first and second fragments of the bone.

8. The method according to claim 1, wherein the identified landmarks are points on the linea aspera of a femur.

9. The method according to claim 8, further comprising:
aligning the linea aspera of the first fragment and the linea aspera of the second fragment relative to each other such that the linea aspera of a healed bone formed from the first and second fragments approximates a straight line.

10. The method according to claim 1, further comprising:
using a navigation system to locate the ultrasound device at a predetermined position, to identify at least one landmark on each of the first fragment of the bone and second fragment of bone.

11. The method according to claim 1, further comprising:
connecting the first fragment and the second fragment of the bone to each other by placing an intramedullary pin into at least a portion of a medullary channel.

12. The method according to claim 11, further comprising:
determining a position of the intramedullary pin using x-ray recordings.

13. The method according to claim 11, further comprising
using a navigation system to locate the intramedullary pin at a predetermined position.

14. The method according to claim 1, wherein the identification of at least one landmark on each of the first fragment of the bone and the second fragment of the bone is automatic.

15. The method according to claim 14, wherein the automatic identification uses image recognition algorithms.

16. The method according to claim 1, further comprising:
displaying guidance for aligning the first and second fragments of the bone.

17. A computer program embodied on a computer readable medium for aligning a first fragment and a second fragment of a fractured bone relative to each other, comprising
code that identifies at least one landmark on each of the first fragment of the bone and second fragment of bone from imaging data of the first and second fragments of the bone;
code that determines a spatial position and/or orientation of each of the identified landmarks; and
code that uses the determined spatial position and/or orientation of each of the identified landmarks to determine an alignment of the first and second fragments of the bone that satisfies a predetermined criteria.

18. A system for aligning a first fragment and a second fragment of a fractured bone relative to each other, said first and second fragments each having a tracking reference attached thereto, comprising:
an ultrasound device including a tracking reference;
a tracking system configured to spatially track the tracking references; and
a computational unit operatively coupled to said tracking system and said ultrasound device, said computational unit comprising
a processor and memory, and
logic stored in the memory and executable by the processor, said logic including
i) logic that identifies at least one landmark on each of the first fragment of the bone and second fragment of bone from imaging data of the fragments of the bone obtained from the ultrasound device,
ii) logic that determines a spatial position and/or orientation of each of the identified landmarks from tracking data provided by the tracking system, and
iii) logic that uses the determined spatial position and/or orientation of each of the identified landmarks to determine an alignment of the first and second fragments of the bone that satisfies a predetermined criteria.

19. The device according to claim 18, further comprising:
a display device which is operably connected to the ultrasound device or the computational unit configured to display ultrasound recordings and/or guidance for aligning the first and second fragments of the bone.

20. The device according to claim 18, further comprising logic to automatically identify the at least one landmark on each of the first fragment of the bone and second fragment of bone.
